(19) United States
(12) Patent Application Publication
(10) Pub. No.: US 2019/0083730 A1
(43) Pub. Date: Mar. 21, 2019

(54) Laryngeal Mask

(71) Applicant: E-PROSPERITY & DEVELOPMENT CO., Ltd., Taipei City (TW)

(72) Inventors: CHEN-CHUN LIAO, Taipei City (TW); JIUN-YI LI, Taipei City (TW); CHENG-SHIH LIEN, Taipei City (TW)

(73) Assignee: E-PROSPERITY & DEVELOPMENT CO., Ltd., Taipei City (TW)

(21) Appl. No.: 16/134,978

(22) Filed: Sep. 19, 2018

(30) Foreign Application Priority Data
Sep. 20, 2017 (TW) 106213998

(51) Int. Cl. A61M 16/04

(52) U.S. Cl. CPC A61M 16/0445; A61M 16/0409; A61M 16/0436; A61M 16/0463

(57) ABSTRACT

A laryngeal mask is provided which has a mask body having a capsule body and a support body, wherein a groove is provided on the top of the support body, and the capsule body is correspondingly disposed on the groove; and a tube body having an airway tube body, wherein one end of the tube body is bonded to the support body, and the bonding portion is provided with an opening communicating with one end of the airway tube body.

### FIELD OF THE INVENTION

The present invention relates to a laryngeal mask, which is characterized in that the mask body of the laryngeal mask is composed of a support body and a capsule body disposed on the support body.

### DESCRIPTION OF PRIOR ART

In current clinical practices, the maintenance and smoothness of the respiratory tract is a critically important condition when anesthesia is performed during a surgical procedure, especially when general anesthesia is performed. For some special patients, such as patients suffering from ankylosing spondylitis and the like, the cervical vertebrae of such patients are usually very stiff, even unable to move, hence it may be very difficult to perform intubation. Currently, there are many ways to maintain an unobstructed respiratory tract. For example, an air way and an endotracheal tube can be used in intubation to maintain the patient's respiratory tract unobstructed during anesthesia.

However, the above-described intubation method may cause trauma to patients. An insertion type laryngeal mask has gradually been widely used in recent years. This type of laryngeal mask is an alternative to conventional respiratory anesthesia that requires trachea intubation where the laryngeal mask is used to maintain the tracheal access. It can be used in general emergency situations to rescue semi-conscious or completely unconscious patients, assisting in smoothing airflow in the airway and performing artificial respiration.

Please refer to FIG. 1 which shows a cross-sectional view of a conventional laryngeal mask. As shown in FIG. 1, an inflatable laryngeal mask 10 mainly comprises a flexible tube body 11, an inflatable mask ring 12 and an inflatable air tube 13. The flexible tube body 11 comprises a first opening part 111 and a second opening part 112, the inflatable mask ring 12 is disposed annularly around the first opening part 111, one end of the inflatable air tube 13 and the inflatable mask ring 12 are in communication with each other, the other end is used to guide gas into the inflatable mask ring 12. When the inflatable mask ring 12 of the inflatable laryngeal mask is inflated, the inflatable mask ring 12 may be partially abutted against by the larynx of a patient due to poor installation of the inflatable mask ring 12. As a result, part of the inflatable mask ring 12 cannot be filled with gas, and the inflatable mask cannot be fully expanded. Further, since the inflatable mask ring 12 is not fully expanded, the inflatable mask ring 12 in not fully in alignment with the laryngopharynx of the patient, a gap is thus generated between the two, resulting in gas leakage and poor ventilation.

Accordingly, because the inflatable mask ring of a conventional laryngeal mask itself does not have any auxiliary supporting device, and the design of its curvature is not in conformance with the oral airway of a human body, when the conventional laryngeal mask is used, it does not tightly seal the laryngopharynx of the human body, resulting in poor gas supply. Therefore, it is necessary to improve the design of the current laryngeal mask.

### DETAILED DESCRIPTION OF THE INVENTION

In view of the foregoing, according to many years of experience, many discussions, trials, embodiment and tests, many revisions and improvements, the inventor of the present invention has created the present invention to solve the above-described problem.

The term “a” or “an” as used herein is to describe elements and components of the present invention. This description should be interpreted as being inclusive of one or at least one, and unless the context clearly dictates otherwise the singular forms “a,” and “an” include the plural forms. When the term “one” is used in conjunction with the term “comprising” in claims, the term “one” may be interpreted as one or more than one.

The term “or” as used herein means “and/or.”

The present invention provides a laryngeal mask which comprises: a mask body having a capsule body and a support body, wherein a groove is provided on the top of the support body, and the capsule body is correspondingly disposed on the groove; and a tube body having an airway tube body, wherein one end of the tube body is bonded to the support body, the bonding portion is provided with an opening communicating with one end of the airway tube body.

In one embodiment, the material of the capsule body, the support body or the tube body is a polymer material. In a preferred embodiment, the polymer material is a soft polymer material having bendability. In another preferred embodiment, the polymer material is a silica gel, polyvinyl chloride, a rubber or a plastic. In addition, the material of the entire laryngeal mask of the present invention is of medical grade; the various components of the laryngeal mask can be further made of a biodegradable material, causing no burden to the human body or the environment. In another embodiment, the support body and the tube body is an integrally formed structure.

The airway tube body of the present invention is in communication with the mask body through an opening at the bonding portion of the support body and the tube body, a medical personnel therefore can input gas from the airway tube body into the respiratory tract of a patient.

In one embodiment, the function of the airway tube body is to input and output gas. In a preferred embodiment, the airway tube body further comprises an airway tube for inputting and outputting gas. Therefore, the airway tube body itself can supply gas for maintaining the patient's autonomous breathing. Alternatively, the airway tube body can serve as a conduit passage, an airway tube can be further provided in the airway tube body to supply gas for maintaining the patient's autonomous breathing. Further, one end of the airway tube body (the end that is not bonded to the support body) may be connected to a respirator to assist patients in breathing.

The top portion of the support body of the present invention is an open end, the groove is substantially disposed annularly around the periphery of the open end; further, the bottom portion of the support body is a closed end. The top surface of the support body is oriented toward the oral airway of a human body when the laryngeal mask is in use. In one embodiment, the support body is substantially an annular body; however, the overall shape is not limited thereto, and may be in an olive shape, an elliptical shape, a circular shape or other geometric shapes. The groove is disposed on the support body, thus the shape of the groove is similar to that of the support body. In a preferred embodiment, the groove is substantially in the shape of a ring. Further, since the capsule body is correspondingly disposed on the groove, the shape of the capsule body substantially corresponds to the shape of the groove, for example, when the groove is substantially in the shape of a ring, the capsule body is also substantially in the shape of a ring, so that the capsule body can be fitted into the groove. Therefore, in another embodiment, the capsule body is substantially in the shape of a ring.

In one embodiment, the manner by which the capsule body is disposed on the groove includes, but is not limited to, adhering, bonding, and fitting.

In another embodiment, the interior of the capsule body has a filler. The filler enables the capsule body to have a specific volume and shape, and the filler can be filled into the capsule body before the laryngeal mask of the present invention is used, so that the capsule body has a specific volume and shape; or when the laryngeal mask is being used (for example, after the laryngeal mask is inserted into the laryngopharynx), the filler is then filled into the capsule body.

The “filler” as described herein includes, but is not limited to, a gas or a liquid. In one embodiment, the filler is a liquid. In a preferred embodiment, the liquid is water or a hydrogel. In a more preferred embodiment, the liquid is a hydrogel. When the filler is a hydrogel, since the hydrogel has a property of being solidified at a specific temperature, after the hydrogel in the capsule body is solidified, the capsule body is adhered to cover the laryngopharynx of a patient more tightly, providing a better airtight effect.

Therefore, in one embodiment, the tube body further comprises a filling tube body, one end of which is connected to the capsule body for inputting and extracting the filler in and out of the capsule body. In a preferred embodiment, the filling tube body comprises a filling tube, one end of which is connected to the capsule body for inputting and extracting the filler in and out of the capsule body. Therefore, the filling tube body itself can provide the function of inputting and outputting the filler in and out of the capsule body. Alternatively, the filling tube body can serve as a conduit passage, a filling tube is further disposed therein, the function of inputting and outputting the filler in and out of the capsule body is provided by the filling tube.

The tube body of the present invention is provided with the airway tube body and the filling tube body, therefore, in one embodiment, the tube body is a two-cavity tube body composed of the airway tube body and the filling tube body arranged side by side in parallel but not in communication with each other.

The support body of the present invention is provided with a filling opening arranged on the groove, and the filling tube body or the filling tube passes through the support body and bonds to the capsule body through the filling opening. Therefore, the capsule body of the present invention is provided with a filling hole, one end of the filling tube body or the filling tube is connected to the capsule body and fitted into the filling hole, so that the capsule body and the filling tube body or the filling tube are connected. A medical personnel can input a filler (such as a gas or a liquid) into the capsule body through the filling tube body or the filling tube to expand the capsule body, as a result, the expanded capsule body tightly adheres to the laryngopharynx of a human body to achieve a good sealing effect. In one embodiment, the filling tube body or the filling tube increases or decreases the amount of the filler in the capsule body by using the input or extraction function to control the volume of the capsule body. In another embodiment, the end of the filling tube body or the filling tube (the end that is not bonded to the capsule body) is provided with a check valve for controlling the input or extraction of the filler.

In addition, the capsule body of the present invention can be designed in such a way that it is filled with a specific amount of filler before the laryngeal mask is inserted into the throat of a patient, so that the capsule body has a specific volume when it is inserted into the patient body to cover the laryngopharynx of the patient. Alternatively, the interior of the capsule body is filled with a small amount of filler or no filler at all before the laryngeal mask is inserted into the throat of the patient, after the laryngeal mask reaches the laryngopharynx of the patient, the filler is then filled into the capsule body through the filling tube body or the filing tube, expanding the capsule body to cover the laryngopharynx of the patient.

In order to achieve a better sealing effect when the laryngeal mask is used clinically, the shape of the mask body is in conformance to the curvature of the oral airway. Since the top surface of the support body of the present invention is the side facing the human throat when the laryngeal mask is used, the curvature of the top surface of the support body is designed to conform to the curvature of the oral airway of the human body. Since the groove is disposed on the top surface of the support body, the groove shows variations in curvature, the capsule body expanded due to the filled liquid or gas and disposed on the groove also shows variations in curvature. When the capsule body conforms to the curvature of the oral airway of human body, the expanded capsule body adheres tightly to the human throat. Therefore, in one embodiment, the top surface of the support body is a curved surface, and the curvature of the curved surface substantially conforms to the curvature of the oral airway of a human body.

Further, when a medical personnel tries to insert the laryngeal mask into a human larynx, the bendability of the flexible tube body on the laryngeal mask may affect the medical personnel's performance and cause discomfort to the patient, so the degree of the bendability of the flexible tube body is critically important. Therefore, in one embodiment, the tube body has a curved segment in the lengthwise direction, and the curvature of the curved segment is adapted for easy insertion of the laryngeal mask into the larynx of a human body. In a preferred embodiment, the curvature of the curved segment of the tube body is 60-80 degrees.

In one embodiment, the tube body further comprises an extraction tube body. The extraction tube body itself can provide the function of extraction esophageal liquid and/or gastric fluid. Alternatively, the extraction tube body can serve as a conduit passage, an extraction tube is further disposed therein, the function of extracting esophageal liquid and/or gastric fluid is provided by the extraction tube. Therefore, in a preferred embodiment, the extraction tube body further comprises an extraction tube for extracting esophageal liquid and/or gastric fluid. In another embodiment, the front end of the support body is provided with an open hole which is in communication with the extraction tube body for allowing the extraction tube to pass. Thus, when a laryngeal mask is used in a medical procedure, the extraction tube can be arranged in and passes through the extraction tube body (which serves as a conduit passage), and eventually passes through the open hole toward the patient's esophagus direction. At this time, the medical personnel can timely extract residual liquids or digestive liquids in the stomach and/or esophagus through the extraction tube. Alternatively, a diversion tube can be used, which passes through the extraction tube body to guide the gastric fluid and/or esophageal liquids to be discharged through the open hole and the opening of the diversion tube.

In another embodiment, the tube body is a three-cavity tube body including the airway tube body, the filling tube body, and the extraction tube body arranged side by side in parallel but not in communication with one another. The tube body of the present invention adopts a design in which three tubes (an airway tube body, a filling tube body and an extraction tube body) are arranged side by side, so that the three tube bodies can respectively communicate with the corresponding tubes or openings of the mask body, the thickness of the adjacent tube walls formed between the three tubes can prevent the tube bodies of the three tubes from being folded to avoid gas blockage. Further, the thickness of the adjacent tube walls formed by the parallel tubes can also be used as a seaming device to effectively prevent patients from biting the tubes to block the gas flow.

The laryngeal mask provided by the present invention, when compared with other conventional techniques, has the following advantages:

(1) The tube body on the laryngeal mask of the present invention has a specific angle, the specific angle facilitates insertion of the laryngeal mask into the throat of a patient. Further, the tube body is a fixed structure, and the tubes/tube lines contained in the tube body adopt a side-by-side design, so that the tubes are not bent or folded back after entering the throat, otherwise, the supplied gas may be blocked, unable to be delivered into the throat, causing a problem of poor gas intake; and

(2) The mask body of the laryngeal mask of the present invention is to be designed with a groove pattern on a support body, and then a capsule body is fitted onto the groove. Such design can provide a support to the capsule body, so that it can stably cover the opening of the throat of a patient. In addition, the top surface of the support body is designed to have a curvature substantially in conformance to the curvature of the oral airway of a human body, the curvature is also shown by the expanded capsule body which is disposed on the top of the support body, allowing the capsule body to be fitted more tightly to the oral airway of the patient. Therefore, the design of the laryngeal mask of the present invention can provide a good airtight effect and avoid the occurrence of gas leakage.

### BRIEF DESCRIPTION OF THE DRAWINGS

FIG. 1 is a schematic view of a conventional laryngeal mask.

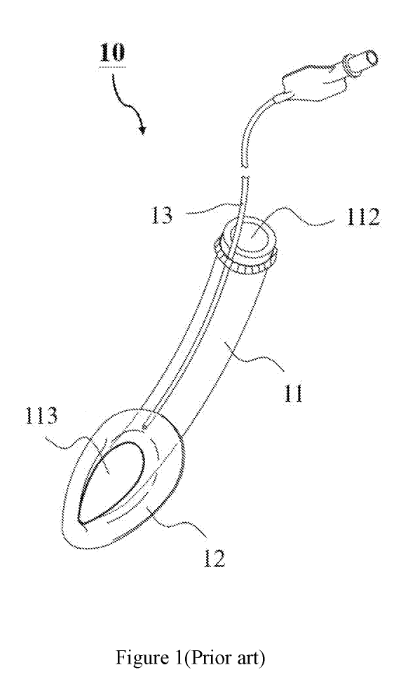

FIG. 2 is a structural diagram of a capsule body-free laryngeal mask of the present invention.

FIG. 3 is a structural diagram of a laryngeal mask having a capsule body of the present invention.

### EXAMPLES

The present invention may be implemented in many different forms and should not be construed as limited to the examples set forth herein. The described examples are not limited to the scope of the present invention as described in the claims.

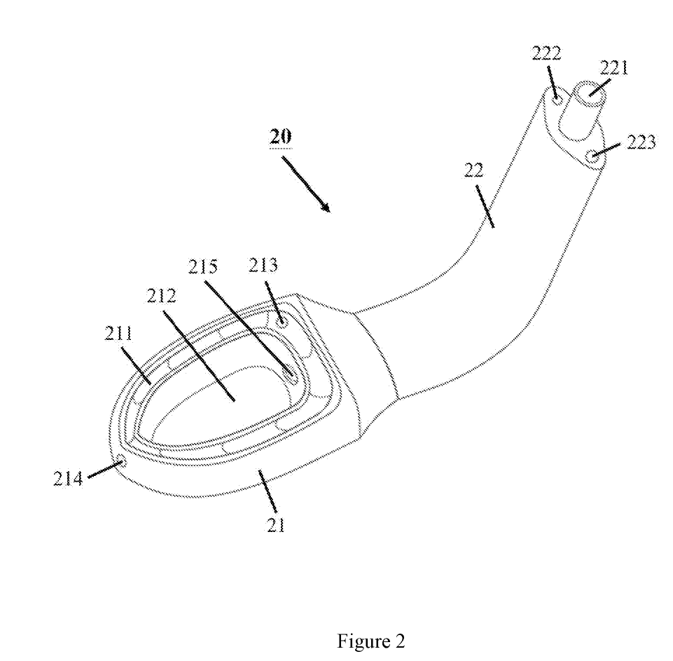

As shown in FIG. 2, it is a schematic structural diagram of a laryngeal mask without a capsule body. The laryngeal mask 20 comprised: a support body 21, wherein a groove 211 was provided on the top of the support body 21, and the top surface of the support body 21 was the side facing the oral airway of a human body when the laryngeal mask was used clinically; and a tube body 22 having an airway tube body 221, a filling tube body 222 and an extraction tube body 223, wherein one end of the tube body 22 was bonded to the support body 21, and the bonding portion thereof was provided with an opening 215 in communication with one end of the airway tube body 221.

An airway port 212 was provided on the top of the support body, the groove 211 was disposed around the periphery of the airway port 212, so that the groove 211 was substantially in the shape of a ring. The support body 21 was used for receiving and supporting a capsule body, thus the capsule body was correspondingly disposed on the groove 211 of the support body 21; the shape of the capsule body substantially corresponded to the shape of the entire groove 211. As a result, the shape of the capsule body was substantially in the shape of a ring. In addition, the groove 211 had a fill-in opening 213 allowing the filling tube body 222 to pass through and bond to the capsule body for the filling tube body 222 to input a filling liquid or gas into the capsule body.

The tube body 22 was a three-cavity tube body including the airway tube body 221, the filling tube body 222, and the extraction tube body 223 arranged side by side in parallel but not in connection with one another. The airway tube body 221 was used for introducing gas into the laryngopharynx of the patient, and the filling tube body 222 served as a conduit passage for a filling tube to be additionally disposed in the filling tube body 222, the filling tube passed through the filling tube body 222 to connect with the capsule body for inputting and extracting a filler (for example, a liquid or a gas) into the capsule body, thereby controlling the volume of the capsule body. In addition, the support body 21 had an open hole 214 which was in communication with the extraction tube body 223. Therefore, the medical personnel was able to insert an extraction tube in the extraction tube body 223, the extraction tube passed through the extraction tube body 223, and eventually passed through the open hole 214, facing towards the direction of esophagus. Therefore, the medical personnel was able to extract residual gastric and esophageal liquids, digestive liquids or gases by using the extraction tube.

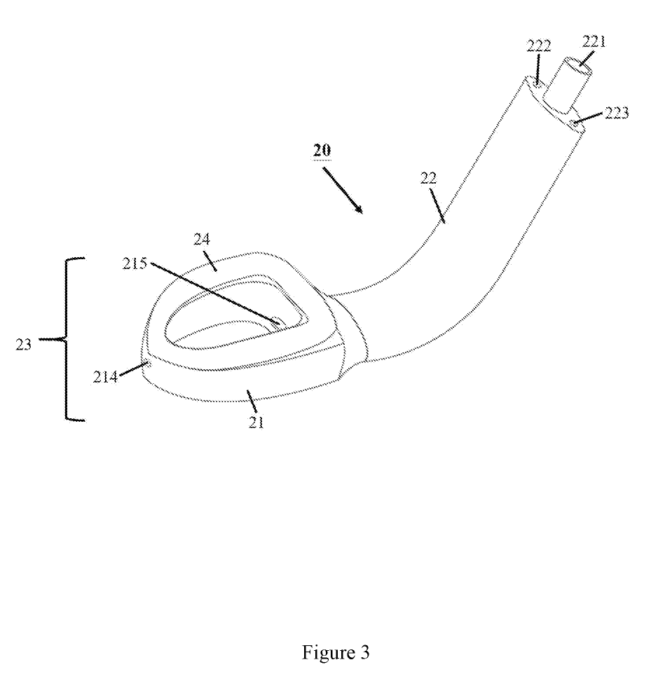

Therefore, a capsule body was further disposed on the groove 211, which constituted the design of the entire structure of the laryngeal mask of the present invention. Therefore, as shown in FIG. 3, it is a schematic structural diagram of the laryngeal mask having a capsule body of the present invention. The laryngeal mask 20 comprised: a mask body 23 having a capsule body 24 and a support body 21, wherein a groove was provided on the top of the support body 21, the capsule body 24 was correspondingly disposed on the groove, wherein the top surface of the support body 21 was the side facing the oral airway of a human body when the laryngeal mask was used clinically; and a tube body 22 having an airway tube body 221, a filling tube body 222 and an extraction tube body 223, wherein one end of the tube body 22 was bonded to the support body, the boding portion thereof was provided with an opening 215 communicating with one end of the airway tube body 221, and one end of the filling tube body 222 was in communication with the capsule body 24.

When the laryngeal mask 20 of the present invention was used clinically, the applications and manners of use were substantially the same as those of a conventional laryngeal mask. The capsule body 24 of the laryngeal mask 20 of the present invention was first unfilled, i.e., unexpanded, the laryngeal mask 20 was gradually extended from a patient's mouth until it reached the larynx. Thus, the tube body 22 of the laryngeal mask of the present invention was designed to have a cured segment in the lengthwise direction, and the curvature of which facilitated the insertion of the laryngeal mask 20 into the patient's larynx. In addition, through the tube body 22, the airway tube body 221, the filling tube body 222 and the extraction tube body 223 were designed to be arranged side by side, so that the airway tube body 221, the filling tube body 222 and the extraction tube body 223 were respectively in communication with the support body 21, and the thickness of the adjacent tube walls formed between the three tubes was able to prevent the tube body of the three tubes from being folded to avoid gas blockage. Further, the thickness of the adjacent tube walls formed by the tubes arranged side by side in parallel could also be used as a seaming device to effectively prevent patients from biting the tubes to block the gas flow.

When the laryngeal mask 20 reached the opening at the laryngopharynx of the patient, the end surface of the mask body 23 having the capsule body 24 was aligned with the opening of trachea at the larynx of the patient, and then a filling tube was allowed to pass through the filling tube body 222 and connected with the capsule body 24, the medical personnel inputted a filler (such as a hydrogel or a gas) into the capsule body 24 through the filling tube, so that the capsule 24 was fully inflated to cover the opening of trachea at the larynx of the patient. In addition, the end of the filing tube body 222 was provided with a check valve for controlling the input or extraction of the filler.

Since the capsule body 24 of the present invention was mounted on the support body 21, the top surface of the support body 21, that is, the side on which the capsule body 24 was mounted, was a curved surface, the curvature of the curved surface was substantially in conformance to the curvature of the oral airway of the human body. Therefore, the groove on the top surface of the support body 21 also had the curvature, as a result, the shape and the curvature of the inflated and expanded capsule body 24 correspondingly disposed on the groove conform to the curvature of the oral airway, and tightly adhere to the laryngopharynx of the patient. Further, the support body 21 could be used as a fixed base to provide a specific supporting force to the capsule body 24, so that the mask body 23 could tightly cover the opening of the laryngopharynx of the patient. Therefore, when the mask body 23 inputted gas through the airway tube body 221, the gas could be stably delivered from the opening 215 at the bonding portion of the tube body 22 and the support body 21 to the oral airway of the patient, causing no gas dissipation.

In addition, the open hole 214 on the support body 21 must align with the esophagus of the patient, so that the medical personnel could pass an extraction tube through the extraction tube body 223 and the open hole 214 towards the esophagus to extract and discharge the residual gastric and esophageal liquids or digestive liquids out of the patient's body.

The above-described examples are preferred embodiments of the present invention only, they are not intended to limit the scope of the present invention, that is, simple and equivalent variations and modifications made according to the claims and the content of the specification of the present invention should be within the scope of the present invention.

What is claimed is:

1. A laryngeal mask, comprising
a mask body having a capsule body and a support body, wherein a groove is provided on the top of the support body and the capsule body is correspondingly disposed on the groove; and
a tube body having an airway tube body, wherein one end of the tube body is bonded to the support body, and the bonding portion is provided with an opening communicating with one end of the airway tube body.

2. The laryngeal mask of claim 1, wherein the support body is substantially an annular body.

3. The laryngeal mask of claim 2, wherein the groove and the capsule body are substantially in the shape of a ring.

4. The laryngeal mask of claim 1, wherein the top surface of the support body is a curved surface and the curvature of the curved surface is substantially in conformance to the curvature of the oral airway of a human body.

5. The laryngeal mask of claim 1, wherein the tube body is provided with a curved segment in the lengthwise direction, and the curvature of the curved segment facilitates insertion of the laryngeal mask into the larynx of a human body.

6. The laryngeal mask of claim 5, wherein the curvature of the curved segment of the tube body is 60-80 degrees.

7. The laryngeal mask of claim 1, wherein the interior of the capsule body is provided with a filler.

8. The laryngeal mask of claim 7, wherein the tube body further comprises a filling tube body, and one end of the filling tube body is connected with the capsule body for inputting and extracting the filler in and out of the capsule body.

9. The laryngeal mask of claim 7, wherein the filler is a gas or a liquid.

10. The laryngeal mask of claim 9, wherein the liquid is a hydrogel.

11. The laryngeal mask of claim 8, wherein the tube body further comprises an extraction tube body.

12. The laryngeal mask of claim 11, wherein the extraction tube body further comprises an extraction tube for extracting esophageal liquid and/or gastric fluid.

13. The laryngeal mask of claim 11, wherein the tube body is a three-cavity tube body including the airway tube body, the filling tube body and the extraction tube body arranged side by side in parallel but not in connection with one another.
